(19) United States
(12) Patent Application Publication
(10) Pub. No.: US 2021/0262008 A1
(43) Pub. Date: Aug. 26, 2021

(54) METHOD OF PERSONALIZED TREATMENT FOR CARDIOMYOPATHY AND HEART FAILURE AND OTHER RELATED DISEASES BY MEASURING RENIN ACTIVITY, PRO-RENIN, PRO-RENIN RECEPTOR LEVELS IN BLOOD

(71) Applicant: ARIZONA BOARD OF REGENTS ON BEHALF OF THE UNIVERSITY OF ARIZONA, Tucson, AZ (US)

(72) Inventors: Guy Reed, Phoenix, AZ (US); Inna Gladysheva, Phoenix, AZ (US); Ryan Sullivan, Phoenix, AZ (US); Ranjana Tripathi, Phoenix, AZ (US)

(21) Appl. No.: 17/313,938

(22) Filed: May 6, 2021

Related U.S. Application Data
(63) Continuation-in-part of application No. PCT/US19/60078, filed on Nov. 6, 2019.
(60) Provisional application No. 62/756,427, filed on Nov. 6, 2018.

(51) Int. Cl. C12Q 1/37; A61K 31/16; G01N 33/573; A61P 9/04

(52) U.S. Cl. CPC C12Q 1/37; A61K 31/16; G01N 33/573; G01N 2800/325; G01N 2800/52; G01N 2333/96483; A61P 9/04

(57) ABSTRACT

Methods of using measured plasma renin activity assayed (PRA), plasma renin activity concentration (PRAC), active renin concentration (ARC), plasma renin activity concentration (PRAC) and/or active plasma renin concentration (APRC), pro-renin activity, (pro)-renin receptor (PRR) levels for diagnosing, prognosing, treating and monitoring heart failure (HF) or HF-associated conditions or other conditions that cause or are caused by altered (e.g., elevated) renin activity, pro-renin levels, PRR levels, PRC, APRC, and/or interaction of renin or pro-renin with PRR. These methods can be used for at-risk patients for personalized therapy of heart disease to reduce heart dysfunction progression, diminish HF, and prolong life. This technology stratifies patients with HF or those at risk for HF to determine the appropriate medication (agents that specifically interfere with plasma renin activity/pro-renin and/or PRR) or intervention and the appropriate dosage of medication to treat further HF progression.

### CROSS-REFERENCES TO RELATED APPLICATIONS

This application is a continuation-in-part and claims benefit of PCT Application No. PCT/US19/60078, filed Nov. 6, 2019, which claims benefit of U.S. Provisional Patent Application No. 62/756,427, filed Nov. 6, 2018, the specifications of which are incorporated herein in their entirety by reference.

### BACKGROUND OF THE INVENTION

Yearly, one in every four deaths in the United States is caused by heart disease, approximately 610,000 deaths. Currently, heart disease is treated with lifestyle changes, medications, or surgery. There is a need for a method of detecting heart disease early on and a treatment plan that is personalized to the condition of the individual patient and can prevent the progression of other ailments that result from heart disease, including heart failure (HF).

### FIELD OF THE INVENTION

The present invention relates to methods of using measured plasma renin activity assayed and defined as PRA, active renin concentration (ARC), and/or active plasma renin concentration (APRC), plasma renin activity concentration (PRAC), or other methods; pro-renin activity, and (pro)-renin receptor (PRR) levels, for diagnosing, prognosing, treating and monitoring HF-associated conditions or other conditions that cause or are caused by altered (e.g., elevated) renin activity, pro-renin levels, PRR levels, PRC, ARC, APRC, and/or interaction of renin or pro-renin with PRR. The methods described herein also can be used for at-risk patients for personalized therapy of heart disease to reduce the progression of heart dysfunction, diminish HF, and prolong life. This technology intends to stratify patients with HF or those at risk for HF, to determine the appropriate medication (agents that specifically interfere with plasma renin activity/pro-renin and/or PRR) or intervention as well as the appropriate dosage of medication to use to help treat further progression of HF.

### BACKGROUND ART

Heart failure (HF) has many causes and HF progression is affected by various pathways, including the sympathetic nervous system, the renin-angiotensin-aldosterone-system (RAAS) and the natriuretic peptide (NP) system. Activation of the angiotensin-aldosterone-system is associated with extracellular fluid and sodium retention (edema), left ventricular dysfunction, and cardiac dilation. Renin is an enzyme that specifically catalyzes the first and rate limiting step in the activation of angiotensin II, which also enhances the secretion of aldosterone. Although pathological plasma renin activity longitudinally increases with HF stages and varies between patient subsets, clinical trials with non-personalized approach have failed to confirm a crucial role for renin activity in pathogenesis of HF.

Patients with HF or those at risk are often treated with the same medications, based on findings from large randomized clinical trials, which tend to homogenize individual differences. Current treatment guidelines for HF broadly recommend therapeutics directed against angiotensin, aldosterone, or neprilysin, without the assessment of these (as) biomarkers, although it is widely known that HF has many etiologies and patients show differences in their biomarker profiles. For example, expression and activation of the enzymes, hormones, and receptors of the RAAS are modulated by a variety of factors including sex.

It is increasingly recognized that there are disparities in treatment outcomes related to a variety of factors such as sex, race, geographic location, disease etiology, and genetics causes. For example, in a mouse model of dilated cardiomyopathy (DCM), with translational relevance to human HF, female mice develop HF at an accelerated rate that is indicated by worsening systolic function, increased natriuretic peptide (NP) levels, lung edema, and reduced survival by comparison to males. Prior to development of HF and other biomarker abnormalities, female mice showed increased plasma renin activity levels, linking elevated plasma renin activity levels in female mice to accelerated, sex-related deterioration in systolic function, HF progression, and early mortality.

There remains a need for improving detection/diagnosis, prognosis, and treatment of cardiomyopathy and HF. This necessitates a solution to identify specific biomarkers to aid in developing targeted treatment plans. Improving the treatment management of HF necessitates a personalized medicine approach, which requires a two-prong technique that relies on 1) identification of specific biomarkers, which allow for earlier diagnoses and classification of a disease profile and, 2) a targeted treatment plan for the individual based on their specific profile (e.g., personalized treatment/precision therapy strategy).

Commercial kits are available that detect markers of cardiovascular disease, but do not typically include renin/plasma renin activity/pro-renin/PRR. Cardiovascular MAP (Myriad RBM) detects several (n=77) analytes (excluding renin) associated with cardiovascular disease. TruSight Cardio Kit (IIlumina) uses next-generation sequencing (NGS) to provide coverage of 174 genes (excluding renin) with known associations to 17 inherited cardiac conditions (ICCs), including cardiomyopathies, arrhythmias, aortopathies, and more.

Plasma Renin Activity has been previously evaluated as a potential predictive marker for cardiovascular disease risk and prognostic marker for individuals with HF or other heart-related diseases. Plasma renin activity levels have been suggestive of cardiovascular outcomes, HF, transition to severe HF, and mortality in limited clinical trials. However, clinical trials designed using a non-personalized approach resulted in negative findings. In addition, women were underrepresented in the clinical trials. In addition, increased plasma renin activity levels did not always correlate with higher mortality and reduction of PRA levels did not always correlate with improved outcomes in certain populations of patients with heart conditions. Biomarkers of HF (excluding renin) have been proposed to determine medication(s) that will help particular patients, and plasma renin level has been proposed as a prognosticator of cardiac remodeling and HF. Although several biomarkers are being evaluated to help guide more precision therapy for HF, renin has not been considered as a biomarker for treatment tailoring and monitoring of cardiovascular diseases, including HF.

### BRIEF SUMMARY OF THE INVENTION

It is an objective of the present invention to provide methods that allow for diagnosing, prognosing, treating, and monitoring conditions that cause or are caused by altered plasma renin activity, in particular, HF-related conditions, as specified in the independent claims. Embodiments of the invention are given in the dependent claims. Embodiments of the present invention can be freely combined with each other if they are not mutually exclusive.

The present invention features use of measured plasma renin activity as assayed by PRA, PRAC, ARC, and/or APRC, or other methods, and/or use of measured pro-renin activity and/or use of measured PRR levels for diagnostic, prognostic, and treatment tailoring and monitoring purposes to aid in the personalized management of patients at risk or with HF. As there remains a need for an individualized/personalized treatment of HF based on identifying specific biomarkers, physicians will be able to use the present invention to recommend personalized treatments (i.e., precision medicine), a feature commonly unavailable to patients with HF. The present invention provides a solution addressing the industry need as earlier diagnoses and classification of disease profiles allow physicians to conduct individualized targeted treatment, thus improving clinical outcomes.

One of the unique and inventive technical features of the present invention is using measured plasma renin activity as assayed by PRA, PRAC, ARC, and/or APRC, or other methods, and/or measured pro-renin activity and/or measured (pro)-renin receptor (PRR) levels for personalized medicine of patients with cardiovascular disease or HF. In particular, this invention utilizes the resultant changes in the plasma renin activity biomarker wherein the resultant changes are indicators (e.g., predictive and prognostic) for personalized treatment strategies, prognosis, personalized treatment monitoring, and disease progression monitoring for conditions that cause altered plasma renin activity. Without wishing to limit the invention to any theory or mechanism, it is believed that using plasma renin activity, analyzed and defined as PRA, PRR, PRAC, and/or ARC, and/or using pro-renin activity, and/or using PRR levels advantageously provides for personalized treatment approaches for conditions that cause altered or increased plasma renin activity, including HF-related conditions.

Further, the prior art teach away from using plasma renin activity assayed as PRA, ARC, PRAC, and/or APRC, or other methods as a biomarker for personalized treatment and management of HF because prior art have failed to confirm a crucial role for plasma renin activity in pathogenesis or progression of HF. The present invention showed that elevated plasma renin activity level is a reliable biomarker of HF such that in female mice with dilated cardiomyopathy (DCM), elevated plasma renin activity was correlated to accelerated, sex-related deterioration in systolic function, HF progression, and early mortality. In addition, renin, plasma renin activity, PRA, prorenin, or PRR are typically not included in cardiovascular panels and have not been indicated as promising biomarkers for treatment tailoring and monitoring of cardiovascular diseases, including HF. Reducing plasma renin activity however occurs at the expense of an increased plasma renin concentration (PRC), which may exert direct effects independent of plasma renin activity through the recently discovered PPR. While active renin is acknowledged to initiate production of both angiotensin II (Ang II) and aldosterone, clinical studies have not rigorously examined plasma renin activity in the pathogenesis of HF. Active renin concentration was recently suggested as a potential indicator for guiding HF with reduced EF (HFrEF) in addition to BNP and New York Heart Association (NYHA) classification.

Additionally, the present invention targeted normalization of elevated PRAC and plasma renin activity analyzed by other methods (e.g., PRA, ARC) by direct renin inhibition using a dose (e.g., oral dose) that does not alter the Ang II-aldosterone axis and renin inhibition surprisingly significantly prolongs life, preserves left ventricular function, reduces edema formation, and delays cachexia/sarcopenia.

The present invention features in vitro methods for determining diagnosis and prognosis of a patient (the patient can be symptomatic or asymptomatic for HF-related conditions) who is suspected or has a condition that causes or caused by altered (e.g., elevated) plasma renin activity, including HF and/or HF-related conditions. In preferred embodiments, the method comprises first measuring plasma renin activity, PRR levels, and/or plasma renin activity assayed and defined as PRA, PRAC, ARC, and/or APRC (or plasma renin activity measured by other methods) in blood from the patient. The patient is then diagnosed and/or prognosed based on the measured plasma renin activity and/or PRR levels and/or measured plasma renin activity as assayed and defined as PRA, PRAC, ARC, and/or APRC (plasma renin activity also can be measured by other methods). A personalized treatment approach for the patient is then determined using the diagnosis and/or prognosis based on the measured analytes (e.g., PRA, PRR levels, PRAC, ARC, APRC, and/or plasma renin activity measured by other methods) as well as the specific clinicopathologic characteristics (e.g., sex, age, prior history of HF-related conditions) of the patient.

The present invention further features a method for treating a patient that has a condition that causes or is caused by altered (e.g., elevated) PRA, including HF and/or HF-related conditions. In preferred embodiments, the method comprises first measuring pro-renin activity, PRR levels, and/or plasma renin activity assayed and defined as PRA, PRAC, ARC, and/or APRC (or plasma renin activity measured by other methods) in blood from the patient. The patient is then prognosed and a risk of progression is determined based on the measured PRA, PRR levels, PRAC, ARC, and/or APRC. A personalized treatment approach for the patient is then determined using the prognosis based on the measured plasma renin activity and/or PRR levels and/or measured plasma renin activity as assayed and defined as PRA, PRAC, ARC, and/or APRC (plasma renin activity also can be measured by other methods) as well as the specific clinicopathologic characteristics (e.g., sex, age, prior history of HF-related conditions) of the patient. The invention features an additional step of administering a therapy based on agents that specifically interfere with renin/plasma renin activity and/or renin/plasma renin activity interaction with PRR (receptor).

The present invention also features an in vitro method for personalized treatment monitoring for a condition that causes or is caused by altered (e.g., elevated) plasma renin activity, including HF and/or HF-related conditions. In preferred embodiments, the method comprises first measuring plasma renin activity, PRR levels, and/or plasma renin activity assayed and defined as PRA, PRAC, ARC, and/or APRC (or plasma renin activity measured by other methods) in blood from the patient over time. The patient is then prognosed and a risk of progression is determined based on the measured plasma renin activity and/or PRR levels and/or measured plasma renin activity as assayed and defined as PRA, PRAC, ARC, and/or APRC (plasma renin activity also can be measured by other methods) at the different timepoints (e.g., two to seven days post-diagnosis, one month post diagnosis, three months post diagnosis, or >three months post-diagnosis as compared to baseline measurements at time of diagnosis). The personalized treatment or approach for the patient can then be changed based on the differences of the measured analytes (e.g., plasma renin activity, PRR levels, PRA, PRAC, ARC, APRC, and/or plasma renin activity measured by other methods) as well as the specific clinicopathologic characteristics (e.g., sex, age, diet, exercise, smoking history, prior history of HF-related conditions) of the patient. In some embodiments, the invention features an additional step, for example, changing the therapy or requiring additional monitoring or another intervention if the patient is progressing.

The present invention further features a method for monitoring disease progression of conditions that cause altered (e.g., elevated) PRA, PRA, including HF and/or HF-related conditions. In preferred embodiments, the method comprises first measuring plasma renin activity, PRR levels, and/or plasma renin activity assayed and defined as PRA, PRAC, ARC, and/or APRC (or plasma renin activity measured by other methods) in blood from the patient over time. The patient is then prognosed and a risk of progression is determined based on the measured plasma renin activity and/or PRR levels and/or measured plasma renin activity as assayed and defined as PRA, PRAC, ARC, and/or APRC (plasma renin activity also can be measured by other methods) at the different timepoints (e.g., two to seven days post-diagnosis, one month post diagnosis, three months post diagnosis, or >three months post-diagnosis as compared to baseline measurements at time of diagnosis). A personalized treatment approach for the patient can then be developed (if patient hasn't started treatment) or changed based on the differences of the measured analytes (e.g., plasma renin activity, PRR levels, PRA, PRAC, ARC, APRC, and/or plasma renin activity measured by other methods) as well as the specific clinicopathologic characteristics (e.g., sex, age, diet, exercise, smoking history, prior history of HF-related conditions) of the patient. In some embodiments, the invention features an additional step, for example, starting a therapy or changing the therapy or requiring additional or more frequent monitoring or another intervention if the patient is progressing or having further increases in plasma renin activity. In other embodiments, if the patient is not progressing and having decreases in plasma renin activity, the personalized treatment may not be changed and/or the frequency of monitoring may be decreased.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, 10, and 1D show heart failure (HF) stages, study design, and effects of aliskiren, a direct renin inhibitor (DRI). FIG. 1A shows a schematic overview of the natural history of HF progression, biomarker changes, and experimental design, in an established model of dilated cardiomyopathy (DCM) in female mice. Female mice with DCM begin to show declines in heart systolic function (ejection fraction; EF) and increases in plasma renin activity (PRA) around 7 weeks of age (Stage B HF), which is prior to the development of progressive edema (Stage C HF), further declines in systolic function, rises in atrial/B-type natriuretic peptide (ANP/BNP) and death. Mice with DCM were randomly treated with aliskiren (DCM+DRI) or nothing (DCM+vehicle) in drinking water (see Examples Section). Vertical hash-mark lines indicate time points for measurement of body composition, while echocardiography and blood-tissue collection were completed at 90 days. FIG. 1B shows the impact of aliskiren treatment on PRA at 90 days. FIG. 1C shows the impact of aliskiren on angiotensin II (Ang II) at 90 days. FIG. 1D shows the impact of aliskiren on aldosterone levels at 90 days. The number of DCM mice is indicated. For reference in FIGS. 1B-1D, values for wild-type (WT) littermates are shown as a dashed line (n=4). Data analyzed with one-way ANOVA and represented as mean±SE. Not significant (NS), ⁺⁺p<0.01, ⁺⁺⁺p<0.001 (solid circle, WT vs. DCM+vehicle; solid square, WT vs. DCM+DRI), *** p<0.001 (DCM+vehicle vs. DCM+DRI).

FIGS. 2A, 2B, 2C, 2D, 2E, 2F, and 2G show that direct renin inhibitor (DRI) treatment significantly improves survival and systolic function in mice with dilated cardiomyopathy (DCM). FIG. 2A shows Kaplan-Meier survival curves of control mice with DCM (DCM+vehicle, red, n=13 deaths+8 censored) vs. DCM mice treated with DRI (DCM+DRI, black, n=21 deaths+8 censored). WT (n=4) values are provided for reference. FIG. 2B shows short axis m-mode examples of DCM+vehicle and DCM+DRI treated mice at 90 days of age. FIG. 2C shows left ventricular systolic function measured as ejection fraction (EF) between DCM+vehicle and DCM+DRI mice. [EF, wildtype (WT)=62.8%]. FIG. 2C shows left ventricular systolic function measured as fractional shortening (FS, WT=34%) between DCM+vehicle and DCM+DRI mice. FIG. 2E shows differences in cardiac output (CO, WT=15.5 mL/min) between DCM+vehicle and DCM+DRI mice. FIG. 2F shows Pearson's correlation analysis of 90-day EF vs. survival. FIG. 2G shows Pearson's correlation analysis of cardiac output (CO) vs. survival. DCM control mice (DCM+vehicle, solid circle, n=20), DCM mice treated with DRI (DCM+DRI, solid square, n=27). Differences between groups were analyzed by Mantel-Cox test and Mann-Whitney test. Pearson's correlation coefficient (rₚ) and p-values are shown. Data are represented as mean±SE, * p<0.05, ** p<0.01 (DCM+vehicle vs. DCM+DRI).

FIGS. 3A, 3B, 3C, and 3D show impact of direct renin inhibitor, aliskiren (DRI-aliskiren) treatment on HF plasma biomarkers in female mice with dilated cardiomyopathy. FIG. 3A shows the impact of aliskiren on atrial natriuretic peptide (ANP) plasma levels at 90 days. FIG. 3B shows the impact of aliskiren on cyclic guanosine monophosphate (cGMP) plasma levels at 90 days. FIG. 3C shows the impact of aliskiren on corin plasma levels at 90 days. FIG. 3D shows the impact of aliskiren on neprilysin plasma levels at 90 days. DCM+vehicle (solid circles) and DRI-aliskiren treated (DCM+DRI, solid squares) group numbers are indicated. For reference, values for wild-type (WT) mice are shown as a vertical dashed line (n=4). Data analyzed with one-way ANOVA and represented as mean±SE. Not significant (NS), +p<0.05, ++p<0.01 (solid circle, WT vs. DCM+vehicle; solid square, WT vs. DCM+DRI) and *** p<0.001 (DCM+vehicle vs. DCM+DRI).

FIG. 4 shows PRAC increases in a sex-dependent manner throughout the course of HFrEF progression in male and female mice with dilated cardiomyopathy (DCM). Wild type males (WT-M): n=5-8/age group; WT females (WT-F): n=6-7/age group; DCM males (DCM-M): n=7-8/age group; DCM females (DCM-F): n=6-8/age group. +/solid square—Difference between WT and DCM females: ++P<0.01, +++P<0.001; +/solid circle—Difference between WT and DCM males: +++P<0.001; */black—Difference between DCM females and DCM males: *** P<0.001. Time-dependent differences between sexes (male vs. female) and differences between genotypes (WT vs. DCM) were analyzed by two-way ANOVA with the Bonferroni posttest correction using GraphPad Prism 8.0.2 (GraphPad Software, San Diego, Calif., USA). Data are expressed as mean±SEM. All animal study activities were approved.

FIG. 5 shows a schematic representation of a potential role of renin activity in the modulation of HFrEF through direct and indirect actions. The plasma renin activity/renin pair directly and indirectly modulates HFrEF progression.

FIG. 6 shows a schematic presentation of assay principles used to measure enzymatic renin activity in plasma samples: Plasma renin activity (PRA); active renin concentration (ARC)/active plasma renin concentration (APRC); and plasma renin activity concentration (PRAC).

FIGS. 7A and 7B show plasma renin activity concentration (PRAC) in healthy control and heart failure (HF) patients with systolic dysfunction. FIG. 6A shows plasma samples of healthy control patients (normal ejection fraction, EF) and patients with reduced (rEF) with and without symptomatic HFrEF. FIG. 6B shows Spearman correlation of PRAC to plasma N-terminal pro-atrial natriuretic peptide (N-ANP). All patients were males and 50-70 years old. Groups were healthy control subjects (n=16), HF with reduced ejection fraction (HFrEF) asymptomatic (n=16), and HFrEF symptomatic (n=15). Venous blood samples were collected using EDTA-aprotinin tubes. This study was approved by the Institutional Review Board, and all subjects gave their informed consent for inclusion before they participated in this study. Data represent mean±SEM. ++P<0.01, (red, Control vs. Asymptomatic)+++P<0.0001 (black, Control vs. Symptomatic), * P<0.05 (Asymptomatic vs. Symptomatic HFrEF). AU=arbitrary units. Comparisons between groups were calculated using the Mann-Whitney test. Statistical analysis was performed with GraphPad Prism 8.0.2 (GraphPad Software, San Diego, Calif., USA). P>0.05 was considered significant.

FIGS. 8A, 8B, 8C and 8D show the effect of cardiac corin-Tg(i) (catalytically inactive corin) overexpression on plasma RAAS biomarkers in female DCM mice. Plasma levels of active renin (arbitrary units, AU) (FIG. 8A), angiotensin II (AngII) (FIG. 8B), aldosterone (Aldo) (FIG. 8C) and angiotensin 1-7 (Ang(1-7)) (FIG. D) in corin-Tg(i)/DCM (⋅,tg,tg) and corin-WT/DCM (⋅,wt,tg) mouse groups, at 90 days of age, n=6-8 per group; dotted line represents control levels in corin-WT/WT (wt,wt) mice. Data are represented as mean±SE, differences were analyzed (for FIGS. 8A, 8B & 8D) by one-way-ANOVA using Newman-Keuls multiple comparison test, and by using Kruskal-Wallis test using Dunn's multiple comparison test (for FIG. 8C). **p<0.01, *p<0.05 (tg,tg or wt,tg vs. wt,wt); ns=non-significant.

FIGS. 9A, 9B, 9C, 9D, and 9E show the dietary sodium restriction activates classical and non-classical RAAS in mice with DCM. The effect of dietary sodium restriction on plasma levels of the classical RAAS: renin activity (AU, arbitrary units) (FIG. 9A), angiotensin II (AngII) (FIG. 9B), aldosterone (FIG. 9C), and counter-regulatory RAAS: angiotensin converting enzyme 2 (ACE2) (FIG. 9D) and angiotensin (1-7) (Ang (1-7)) (FIG. 9E) were determined. Number of DCM mice n=7-8 per group. WT control mice (dashed line, n=5-8) at 17 weeks of age. Data are presented as mean±SEM. ****p<0.0001, ***p<0.001, *p<0.001 (DCM vs. WT); ++++p<0.0001, ++p<0.01, +p<0.05 (DCM on NSD (normal sodium diet) vs. DCM on LSD (low sodium diet)) by one-way ANOVA with Newman-Keuls multiple comparison test.

### DETAILED DESCRIPTION OF THE INVENTION

### Acronyms

ANP: atrial natriuretic peptide

APRC: active plasma renin concentration

ARC: active renin concentration activity

BNP: B-type natriuretic peptide

cGMP: cyclic guanosine monophosphate

CO: cardiac output

DCM: dilated cardiomyopathy

DRI: direct renin inhibitor

EF: ejection fraction

HF: heart failure

HFrEF: heart failure reduced ejection fraction

HFpEF: heart failure preserved ejection fraction

NP: natriuretic peptide

NYHA: New York Heart Association

PRA: plasma renin activity

PRAC: plasma renin activity concentration

PRR: pro-renin receptor

RAAS: renin-angiotensin-aldosterone-system

WT: wildtype

As used herein, the term “cardiovascular disease” refers to conditions of the heart including structural and functional abnormalities. Non-limiting examples comprise: heart failure (HF; a progressive heart disease that affects pumping action of the heart muscles as described herein); tachycardia (a heart rhythm disorder with heartbeats faster than usual, greater than 100 beats per minute in people); cardiomyopathy (an acquired or inherited disease of the heart muscle which makes it difficult for the heart to pump blood to other parts of the body); coronary artery disease (a condition where the major blood vessels supplying the heart are narrowed); angina (chest discomfort or shortness of breath caused when heart muscles receive insufficient oxygen-rich blood); ventricular tachycardia (fast heart beat rhythm of the ventricles), myocardial infarction (death of heart muscle caused by a loss of blood supply); congenital heart defect (abnormality in the heart that develops before birth); atrial fibrillation (a disease of the heart characterized by irregular and often faster heartbeat); ventricular fibrillation (a serious heart rhythm problem in which the heart beats quickly and out of rhythm).

As used herein, “administering” and the like refer to the act of physically delivering a composition or other therapy (e.g. a DRI, e.g., aliskiren) described herein into a subject by such routes as oral, mucosal, topical, transdermal, suppository, intravenous, parenteral, intraperitoneal, intramuscular, intralesional, intrathecal, intranasal or subcutaneous administration. Parenteral administration includes intravenous, intramuscular, intra-arterial, intradermal, subcutaneous, intraperitoneal, intraventricular, and intracranial administration. When a disease, disorder or condition, or a symptom thereof, is being treated, administration of the substance typically occurs after the onset of disease, disorder or condition or symptoms thereof. When a disease, disorder or condition, or symptoms thereof, are being prevented, administration of the substance typically occurs before the onset of the disease, disorder or condition or symptoms thereof.

As used herein, the terms “subject” and “patient” are used interchangeably. As used herein, a subject can be an animal (amphibian, reptile, avian, fish, or mammal) such as a non-primate (e.g., cows, pigs, horses, cats, dogs, rats, etc.) or a primate (e.g., monkey, ape and human). In specific embodiments, the subject is a human. In one embodiment, the subject is a mammal (e.g., a human, a dog) having a disease, disorder or condition described herein. In another embodiment, the subject is a mammal (e.g., a human, a dog) at risk of developing a disease, disorder or condition described herein. In certain instances, the term patient refers to a human under medical care or animals under veterinary care.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “effective amount” as used herein refers to the amount of a therapy or medication (e.g., DRI provided herein) which is sufficient to reduce and/or ameliorate the severity and/or duration of a given disease, disorder or condition and/or a symptom related thereto. This term also encompasses an amount necessary for the reduction or amelioration of the advancement or progression of a given disease (e.g., cardiovascular), disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of an DRI described herein is an amount sufficient enough to provide a therapeutic benefit in the treatment or management of a cardiovascular disease, or to delay or minimize one or more symptoms associated with the presence of the cardiovascular disease. A therapeutically effective amount of an agent (e.g., DRI) described herein, means an amount of therapeutic agent, alone or in combination with other therapies, which provides a therapeutic benefit in the treatment or management of the cardiovascular disease. The term “therapeutically effective amount” can encompass an amount that improves overall therapy, reduces or avoids symptoms or causes of cardiovascular disease, or enhances the therapeutic efficacy of another therapeutic agent.

A therapy is any protocol, method and/or agent that can be used in the prevention, management, treatment and/or amelioration of a given disease, disorder or condition. In certain embodiments, the terms “therapies” and “therapy” refer to a drug therapy, biological therapy, supportive therapy, radiation therapy, and/or other therapies useful in the prevention, management, treatment and/or amelioration of a given disease, disorder or condition known to one of skill in the art such as medical personnel.

Companion Diagnostic (CDx) assays, as defined by the FDA, are in vitro diagnostics (IVD) devices that provide information essential for the safe and effective use of a corresponding therapeutic product. The FDA specifies three main areas where a CDx assay is essential: 1) Identify patients who are most likely to benefit from a particular therapeutic product; 2) Identify patients likely to be at increased risk of serious adverse reactions as a result of treatment with a particular therapeutic product; and 3) To monitor response to treatment for the purpose of adjusting treatment (e.g., schedule, dose, discontinuation) and to achieve improved safety or effectiveness. A CDx can be used both to predict outcome (efficacy and safety) and to monitor response. The FDA has approved or cleared 35 CDx devices (as of 2018), which are available for the treatment of specific leukemias, gastrointestinal tumors, breast cancers, ovarian cancers, melanomas, lung cancers, and colorectal cancers, while no approved CDx assays are available for the treatment of HF, HF-associated and non-associated-sarcopenia/cachexia, -necrosis, -liver disease, -kidney disease, HFrEF, or any condition or treatment that causes or caused by altered plasma renin activity, prorenin or PRR levels.

As described herein, the term “asymptomatic” may refer to a subject without a condition as described herein. In another embodiment, the term “asymptomatic” may refer to a patient diagnosed with a condition (e.g. heart failure) but currently has no symptoms of the condition. In further embodiments, the term “asymptomatic” may refer to a patient diagnosed with a condition (e.g. heart failure) but has responded to previous therapy and displays no symptoms of said condition.

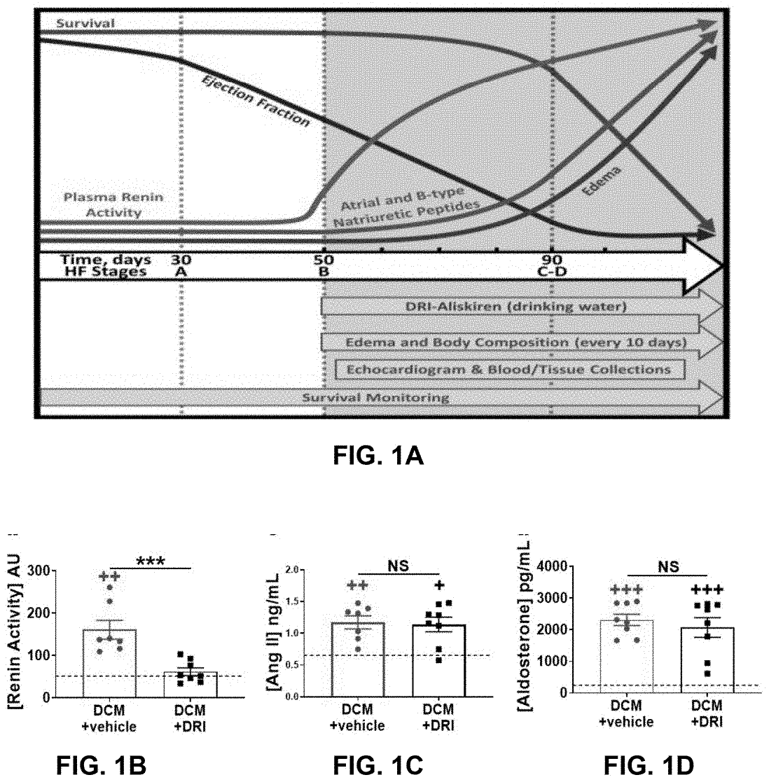

Referring now to FIGS. 1A-9E, the present invention features a new method for developing personalized treatment strategies for conditions that cause or are caused by altered plasma renin activity and/or renin/plasma renin activity interaction with PRR using plasma renin activity for diagnostic purposes. This technology allows for a method to use plasma renin activity to classify disease profiles for prognosis and treatment tailoring and monitoring strategies for disease conditions that cause or are caused by altered PRA and/or renin/plasma renin activity interaction with PRR. In some embodiments, the present invention uses changes in plasma renin activity as indicators for HF prognosis, disease progression, and personalized treatment and monitoring strategies.

Non-limiting examples of the advantages of this technology comprise: 1) personalized medicine strategies for optimal individual treatment for heart disease, HF and/or HF-related conditions; 2) ability to stratify patients with HF or those at risk for HF; 3) preventing progression of systolic heart disease; and 4) treat heart disease.

The present invention features a method for treating a patient who is suffering from a heart condition. In some embodiments, the method comprises determining if the patient has increased plasma renin activity by 1: obtaining a blood sample from the patient and measuring plasma renin activity, and/or measuring pro-renin activity and/or measuring (pro)-renin receptor (PRR) levels and 2: determining a diagnosis or prognosis of said condition based on measured PRA, PRAC, ARC, and/or APRC, and/or measured pro-renin activity and/or measured PRR levels. In some embodiments, plasma renin activity comprises plasma renin activity (PRA), plasma renin activity concentration (PRAC), active renin concentration (ARC), and/or active plasma renin concentration (APRC). In other embodiments, plasma renin activity is assayed and defined as plasma renin activity (PRA), plasma renin activity concentration (PRAC), active renin concentration (ARC), and/or active plasma renin concentration (APRC). In some embodiments, if the patient has an increase of plasma renin activity of more than 5% compared to a baseline level, a treatment is administered to the patient.

The present invention may also feature a method of monitoring effectiveness of a treatment in a patient with a heart condition. In some embodiments, the method comprises determining if the patient has increased plasma renin activity by 1: obtaining a blood sample from the patient and measuring plasma renin activity and/or measuring pro-renin activity and/or measuring (pro)-renin receptor (PRR) levels and 2: determining a diagnosis or prognosis of said condition based on measured PRA, PRAC, ARC, and/or APRC, and/or measured pro-renin activity and/or measured PRR levels. In some embodiments, plasma renin activity comprises plasma renin activity (PRA), plasma renin activity concentration (PRAC), active renin concentration (ARC), and/or active plasma renin concentration (APRC). In other embodiments, plasma renin activity is assayed and defined as plasma renin activity (PRA), plasma renin activity concentration (PRAC), active renin concentration (ARC), and/or active plasma renin concentration (APRC). In some embodiments, monitoring the effectiveness of the treatment in the patient with a heart condition is determined by changes in plasma renin activity compared to a baseline level. In some embodiments, if the patient has an increase of plasma renin activity of more than 5% compared to a baseline level, a different treatment is administered to the patient. In other embodiments, wherein if the patient has a decrease of plasma renin activity more than 5% compared to a baseline level, the patient maintains the same treatment.

In some embodiments, the heart condition comprises heart failure (HF), HF-associated and non-associated-sarcopenia/cachexia, -necrosis, -liver disease, and/or -kidney disease, HF-reduced ejection fraction (HFrEF), HF-preserved ejection fraction (HFpEF), heart dysfunction.

In some embodiments, if the patient has an increase of plasma renin activity of more than about 5% compared to a baseline level, a treatment is administered to the patient. In some embodiments, if the patient has an increase of plasma renin activity of more than about 6% compared to a baseline level, a treatment is administered to the patient. In some embodiments, if the patient has an increase of plasma renin activity of more than about 7% compared to a baseline level, a treatment is administered to the patient. In some embodiments, if the patient has an increase of plasma renin activity of more than about 8% compared to a baseline level, a treatment is administered to the patient. In some embodiments, if the patient has an increase of plasma renin activity of more than about 9% compared to a baseline level, a treatment is administered to the patient. In some embodiments, if the patient has an increase of plasma renin activity of more than about 10% compared to a baseline level, a treatment is administered to the patient. In some embodiments, if the patient has an increase of plasma renin activity of more than about 12% compared to a baseline level, a treatment is administered to the patient. In some embodiments, if the patient has an increase of plasma renin activity of more than about 15% compared to a baseline level, a treatment is administered to the patient.

In some embodiments, a plasma renin activity of more than 20% above baseline indicates a pathological value. In some embodiments, a plasma renin activity of greater than 20% above baseline in combination with other diagnostic tools may be used to diagnose a patient with a condition described herein.

In some embodiments, if the patient has a decrease of plasma renin activity of about 5% compared to a baseline level, a treatment is administered to the patient. In some embodiments, if the patient has a decrease of plasma renin activity of about 6% compared to a baseline level, a treatment is administered to the patient. In some embodiments, if the patient has a decrease of plasma renin activity of about 7% compared to a baseline level, a treatment is administered to the patient. In some embodiments, if the patient has a decrease of plasma renin activity of about 8% compared to a baseline level, a treatment is administered to the patient. In some embodiments, if the patient has a decrease of plasma renin activity of about 9% compared to a baseline level, a treatment is administered to the patient. In some embodiments, if the patient has a decrease of plasma renin activity of about 10% compared to a baseline level, a treatment is administered to the patient. In some embodiments, if the patient has a decrease of plasma renin activity of about 12% compared to a baseline level, a treatment is administered to the patient. In some embodiments, if the patient has a decrease of plasma renin activity of about 15% compared to a baseline level, a treatment is administered to the patient.

In some embodiments, a treatment is administered to a patient until plasma renin activity levels reach normalization. As used herein, “normalization” refers to age and sex matched range (median+/−SD) for healthy/non-diseased population. In other embodiments, the treatment administered to the patient is the same treatment, at a higher lower dose.

In some embodiments, the treatment administered to the patient is a different treatment. In other embodiments, the different treatment administered to the patient is the same treatment, at a higher dose. In some embodiments, the patient is administered the same treatment in combination with another treatment. In other embodiments, the treatment administered to the patient is the same treatment, at a higher lower dose.

In some embodiments, the patient has no increase of plasma renin activity compared to a baseline level then the treatment administered to the patient is the same treatment.

Clinicians are restricted to ACC/AHA Guidelines, hospital protocols, and general best practices for administering or prescribing maximum therapeutic doses. Without wishing to limit the present invention to any theories or mechanisms it is believed that, heart failure patients may develop a tolerance or lack of effect to a particular therapy (e.g., ACE inhibitors) overtime. Changes in medications or treatment strategies should be initiated when there is a lack of response to pathological values of PRA (>20% normal) despite having reached maximum therapeutic dose(s). When this occurs, clinicians should pivot therapy to a different class of drug either independently or synergistically with the original therapy. The goal is to normalize PRA levels and titrate therapeutic doses to the lowest effective dose in order to minimize side effects. Patients can be measured longitudinally to adjust therapies on an as needed basis, resulting in a personalized medicine approach to heart failure treatment.

In appropriate circumstances, the condition that may in part caused by altered plasma renin activity and/or renin/plasma renin activity interaction with PRR comprises HF, HF-associated and non-associated sarcopenia/cachexia, necrosis, liver disease, kidney disease, HFrEF, or any condition or treatment that causes or caused by altered plasma renin activity, prorenin or PRR levels. In preferred embodiments, HF comprises HF-associated and non-associated-sarcopenia/cachexia, -necrosis, -liver disease, and/or -kidney disease, HFrEF, HF with preserved ejection fraction (HFpEF), ascites, organ hypoperfusion, shock, and/or cardiovascular disease. In some embodiments, plasma renin activity comprises the expression and/or activity of renin pathway family members and measuring plasma renin activity comprises measuring the expression and/or activity of renin pathway family members.

In some embodiments, the present invention may be used in combination with other diagnostic methods to accurately diagnose a patient with a condition that causes or is caused by plasma renin activity. In some embodiments, plasma renin activity assayed and defined as plasma renin activity (PRA), plasma renin activity concentration (PRAC), active renin concentration (ARC), and/or active plasma renin concentration (APRC), and/or measuring pro-renin activity and/or measuring (pro)-renin receptor (PRR) levels are biomarkers that may be used in combination with Echocardiography, cMRI, and other clinical factors to help confirm the Stage of HF. In other embodiments, plasma renin activity helps determine the stage of progression for a particular condition described herein.

The present invention further features that the expression and/or activity of renin pathway family members are measured longitudinally. Changes in the expression and/or activity of renin pathway family members are detected longitudinally and longitudinal levels can be compared to baseline levels. In some embodiments, pro-renin level, plasma renin activity, PRA, PRR, PRAC, ARC, and/or APRC measurement is performed in asymptomatic individuals, wherein asymptomatic individuals are individuals without the condition. In appropriate circumstances, baseline levels are normal levels from an aggregate population of asymptomatic individuals without the condition. In other circumstances, baseline levels are relative baseline levels at the time of initial diagnosis of the condition. In preferred embodiments, PRA can be measured at various times, longitudinally, throughout progression of the condition. Non-limiting examples of the timing comprise: 1) at time of diagnosis or initial presentation of symptoms; 2) 12 hours post-diagnosis; 3) two to seven days post-diagnosis, 4) one month post-diagnosis, 5) three months post-diagnosis, or 6) >three months post-diagnosis. In some embodiments, the change in pro-renin level, plasma renin activity, PRA, PRR, PRAC, ARC, and/or APRC is 5% to 10% of baseline, 10% to 20% of baseline, 20% to 50% of baseline, or >50% of baseline. In some embodiments, the change in lean muscle mass level is 5% to 10% of baseline, 10% to 20% of baseline, 20% to 50% of baseline, or >50% of baseline. In some embodiments, the change in fat level is 5% to 10% of baseline, 10% to 20% of baseline, 20% to 50% of baseline, or >50% of baseline.

In some embodiments, the therapeutically effective drugs comprise direct and indirect inhibitors to the renin pathway, which result in decreased renin/pro-renin activity and/or renin/pro-renin interaction with PRR without affecting the Ang II-aldosterone axis. A non-limiting example of the dose range for DRIs comprises 75-600 mg/person, preferably 300 mg/person dose by historical human patient hypertension trials. In other embodiments, the dose of renin inhibitor without affecting the Ang II-aldosterone axis is based on the individual patient; for example, in animals the dose is <50 mg/kg; while not provided for humans in the current literature, doses as low as 75 mg/person have been shown to modulate blood pressure in people. In other embodiments, the therapeutically effective drugs comprise diuretics (e.g., carbonic anhydrase inhibitors), dobutamine, epinephrine, vasodilators, therapeutics against angiotensin, aldosterone or neprilysin, which may be used in combination with direct and/or indirect renin inhibitors. Non-limiting examples of interventions comprise additional or more frequent monitoring and lifestyle changes, for example in diet, exercise, smoking, etc. In some embodiments, other treatments for HF comprise devices that improve or stabilize cardiac function such as pacemakers, defibrillators, circulatory assist devices, artificial hearts and/or heart transplantation.

In some embodiments, the therapeutically effective drugs for heart dysfunction and HF comprise diuretics (e.g. chlorothiazide, HCTZ lasix), beta blockers, Angiotensin converting enzyme inhibitors (ACE inhibitors), Angiotensin II receptor blockers (ARBs), Angiotensin Receptor-Neprilysin Inhibitors (ARNi), Mineralocorticoid Receptor Antagonists (MRA), direct renin inhibitors (DRI), or Sodium-glucose co-transporter-2 (SGLT2) inhibitors.

In other embodiments, the methods can be used to personalize initiation and continuation of therapy based on personal clinicopathologic characteristics of the specific patient. Non-limiting examples of personal clinicopathologic characteristics comprise the measured plasma renin activity assayed and defined as PRA, PRAC, ARC, PRAC and/or APRC or other methods, pro-renin activity, PRR levels, extracellular water content, total water content, lean muscle, fat mass, heart rate, heart rhythm, sex, age, history of diet, exercise, smoking, and/or heart dysfunction specific to the patient.

In preferred embodiments, plasma renin activity is assayed and defined as PRA, PRAC, ARC, PRAC and/or APRC or other methods; and pro-renin activity PRR levels are measured using standard laboratory assays described herein.

In some embodiments, the present invention features a method for prolonging life. Non-limiting examples of prolonging life comprise prolonging life by at least 1 month, at least 3 months, at least 6 months, or at least 1 year or greater, for personalizing initiation and continuation of therapy, for determining the onset of the condition (e.g., HF, liver disease, kidney disease, muscle/fat-wasting or cachexia/sarcopenia). In appropriate circumstances, the method is for reducing the progression of heart dysfunction and diminishing HF, delaying the transition from heart dysfunction to clinical HF, as well as for a self-monitoring method and/or self-modifying of treatments. Other embodiments of the present invention may feature a method that can be used as a Companion Diagnostic for a specific treatment for a condition that causes or is caused by elevated plasma renin, heart dysfunction, HF, and/or a HF-related condition. Another embodiment may feature a method for stratifying patients with HF or those at risk for HF to determine appropriate medication or therapeutic intervention(s) and dosage of medication or augmentation to intervention(s) to treat further progression of HF.

### EXAMPLES

The following are non-limiting examples of practicing the present invention. It is to be understood that the invention is not limited to the examples described herein. Equivalents or substitutes are within the scope of the invention.

### Methods:

Mice: In the Examples 1-3 below, the mice used were all female littermates with or without dilated cardiomyopathy (DCM) on a C57BL/6 background. DCM is a major cause of HF, which is associated with pathological dilation of the heart ventricles and declines in heart contractile or systolic function. DCM mice express transgene dominant negative CREB transcription factor, specific to cardiomyocytes and develop a consistent progressive DCM and HF similar to humans. In female mice with DCM renal function remains within normal range up to the terminal HF stage, as measured by plasma BUN and creatinine. DCM mice (mCREBTg) are slightly hypotensive at 91 days of age compared to mCREB WT. mCREB mice were randomly assigned to experimental groups. Groups were denoted as WT (n=20), DCM (Control, n=28), and DCM+direct renin inhibitor treatment (+DRI, n=29). At 90-day censorship, dissected organs were weighed, and terminal blood was collected via cardiocentesis with EDTA-aprotinin syringes to prevent proteolysis of targeted proteins. The blood samples were centrifuged at 3000 rpm for 20 min at 4° C. Plasma samples were aliquoted and stored at −80° C. until analysis.

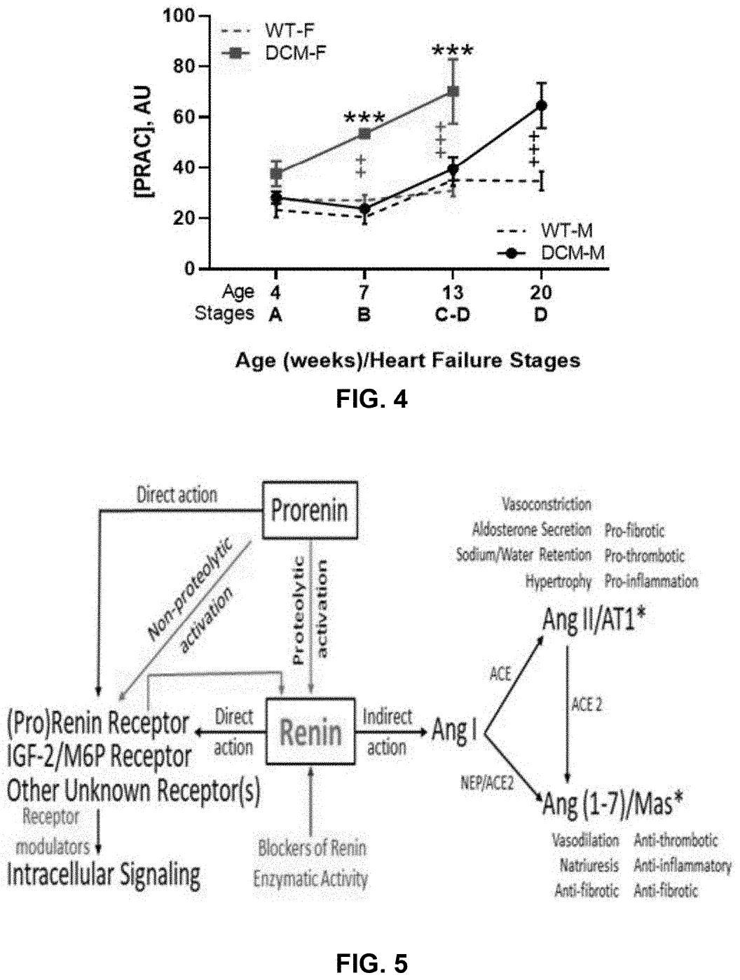

In Example 4 below, female and male mice were used as indicated in FIG. 4 description. The direct renin-inhibitor group (DCM+DRI) was administered aliskiren hemifumarate (BOC Sciences, Shirley, N.Y., USA) at 100 mg/kg/day orally via single source drinking-water in autoclaved hanging bottles. Dose calculations were based on an average consumption of 5 mL of water/day/mouse. The bioavailability of aliskiren is low in humans and only 2.6% orally in rats. Previous studies administering 15-50 mg/kg/day via subcutaneous osmotic pumps showed no alteration in blood pressure. The oral dose was chosen to closely mimic the human route of delivery, while not altering the blood pressure, associated with elevated plasma Ang II-aldosterone levels, given the low bioavailability of the drug in rodents. To prepare the solution, aliskiren powder was dissolved by shaking in volume measured autoclaved 3 ppm hyperchlorinated facility water, mixed fresh every 72 h and shaken daily to resuspend. No issues with palatability or clinical dehydration were noted throughout the study as assessed by blinded husbandry and veterinary staff. There were no side effects or abnormal clinical observations in the mice throughout the treatment period. The aliskiren in drinking water was well tolerated and consumed at expected levels for plain water. The administration was started at 50 days of age to coincide with the previously identified timeline of increased renin activity and development of stage B HF in female DCM mice]. Aliskiren water was provided ad libitum throughout life. All untreated mice (WT and DCM+vehicle) received ad lib 3 ppm hyperchlorinated facility water via an automated watering system (Edstrom, Waterford, Wis., USA).

Echocardiography: The standard transthoracic exam was performed using a Vevo 2100 Imaging System (VisualSonics; Toronto, ON, Canada) with a 30 MHz transducer (MS 400). All imaging was performed at 90 days of age (mice were treated for 35 days before subjecting to echocardiogram). Briefly, mice were induced with 3-5% isoflurane in oxygen and fur removed with depilatory cream (Nair, Church & Dwight Co. Inc., Princeton, N.J., USA). Maintenance anesthesia was held at 2% isoflurane in oxygen throughout the two-dimensional and M-mode recordings of the left ventricle (LV) in parasternal long-axis, short-axis, and four-chamber views. Mouse physiology was maintained at an anesthetized heart rate of 450±50 beats per minute and 37±1° C. rectal temperature. The analysis was blindly completed post-recording using Vevo LAB software (3.1.0, VisualSonics) with three cardiac cycles traced to produce mean values. Ejection fraction (EF, %), fractional shortening (FS, %), LV mass corrected (LVMc, mg), and cardiac output (CO, mL/min) were calculated using standard equations within the software.

Enzyme Immunoassay: Plasma Ang II, aldosterone, atrial natriuretic peptide (N terminus-ANP), cyclic guanosine monophosphate (cGMP), neprilysin, and corin levels were measured by enzyme immunoassays according to the manufacturers' protocols (Phoenix Pharm. Inc., Burlingame, Calif., USA; Abcam Inc., Cambridge, Mass., USA; Enzo Life Sciences Inc., Farmingdale, N.Y., USA; Boster Biological Technology, Pleasanton, Calif., USA; USCN Life Science Inc., Houston, Tex., USA).

Plasma Renin Activity Assay: Renin enzymatic activity from EDTA-aprotinin supplemented mouse plasma samples were measured in a 96-well microplate (Synergy HT reader and Gen5 v1.09 software, BioTek Instruments, Inc., Winooski, Vt., USA) and quantified using exogenous fluorescence resonance transfer (FRET) peptide substrates of renin FRET-QXL™520/5-FAM, optimized for mouse renin (SensoLyte® 520 mouse renin assay kit, AnaSpec, Fremont, Calif., USA). Cleavage of the FRET substrate by mouse renin results in the recovery of quenched fluorescence of 5-FAM, which was detected at excitation/emission=490/520 nm with minimum autofluorescence of plasma samples. The 5-FAM fluorescent reference standard curve was used for results quantification. It is important to note that the PRAC assay differs from PRA and ARC/APRC, which have been historically used to report active renin in clinical trials.

Statistical Analysis: Statistical analyses were performed with GraphPad Prism 7.04 software (GraphPad Software, La Jolla, Calif., USA) using Student's t-test, one-way ANOVA, or two-way ANOVA with Tukey's multiple comparisons test (unless otherwise indicated) and Pearson's correlation. Survival was analyzed using Kaplan-Meier curves with the Mantel-Cox test. Differences were considered significant if p≤0.05. The number of animals (n) is indicated in the figures or figure legends. Data are represented as mean±SE.

### Example 1: Suppression of Elevated Plasma Renin Activity in Females with Dilated Cardiomyopathy (DCM)

FIG. 1A shows HF development from Stage A (no HF), to Stage B (structural heart disease), through Stage C (edema, symptoms), Stage D (severe HF) and death. Female mice with DCM begin to show declines in heart systolic function (ejection fraction; EF) and increases in PRA around 7 weeks of age (Stage B HF), which is prior to the development of progressive edema, further declines in systolic function, rises in atrial/B-type natriuretic peptide (ANP/BNP) and death (FIG. 1A). Female littermate mice with DCM were randomly assigned to receive no treatment (control) or the direct renin inhibitor (+DRI) aliskiren. Treatment with the DRI significantly reduced elevated PRA to normal levels as expected (P<0.01, FIG. 1B). Pathologically elevated plasma aldosterone levels were not modulated by treatment (FIG. 1C). The aldosterone to renin ratio was significantly increased in +DRI mice vs. controls (P<0.05, FIG. 1D).

### Example 2: Renin Activity Suppression Prolongs Survival and Delays Progression of Left Ventricular Systolic Dysfunction

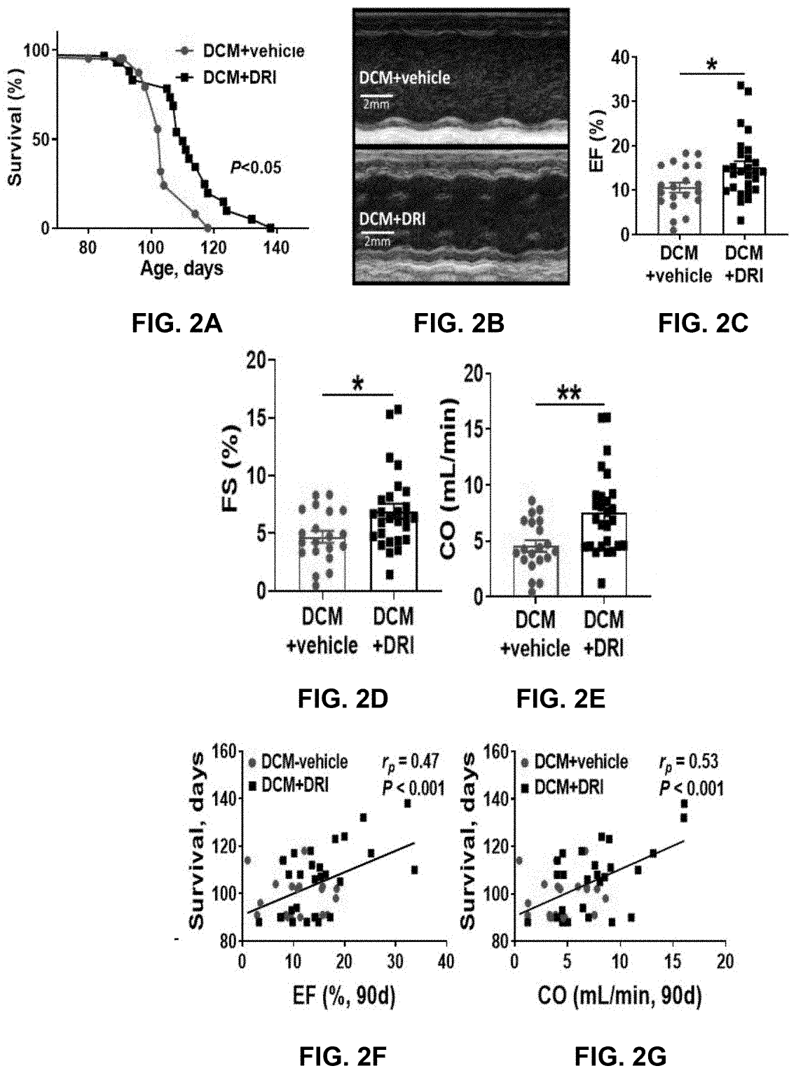

The effect of plasma renin activity suppression was assessed in female mice with DCM as they pass progressively through the stages of HF development from Stage A (no HF), to Stage B (structural heart disease), through Stage C (edema, symptoms), Stage D (severe HF) and death. Three groups of female littermates were examined—DCM control, DCM+DRI and WT mice. WT littermates had 100% survival throughout the 140 day study (data not presented). +DRI mice outlived the control mice by 7% (median survival—110 vs. 103 days respectively, P<0.05, FIG. 2A). In the same experimental groups, cardiac structure and function were assessed by echocardiography at 90 days (Stage C HF with respect to control group). Systolic function in control mice was improved with DRI treatment as measured by ejection fraction (EF %, P<0.05, FIG. 2B) and fractional shortening (FS %, P<0.05, FIG. 2F). Cardiac output (CO mL/min) was also improved with DRI treatment (P<0.01, FIG. 2C), reflecting changes in both heart rate (control 419±10 bpm vs. +DRI 469±14 bpm, P<0.01) and changes in stroke volume (control 11±1 μL vs. +DRI 16±1 μL, P<0.05). Contractile function assessed at 90 days by EF (rₚ=0.47. P<0.001, FIG. 2D) and CO (rₚ=0.53, P<0.05, FIG. 2E) were positively correlated with survival outcome.

### Example 3: Heart Failure Plasma Biomarkers Independently Respond to DRI Treatment

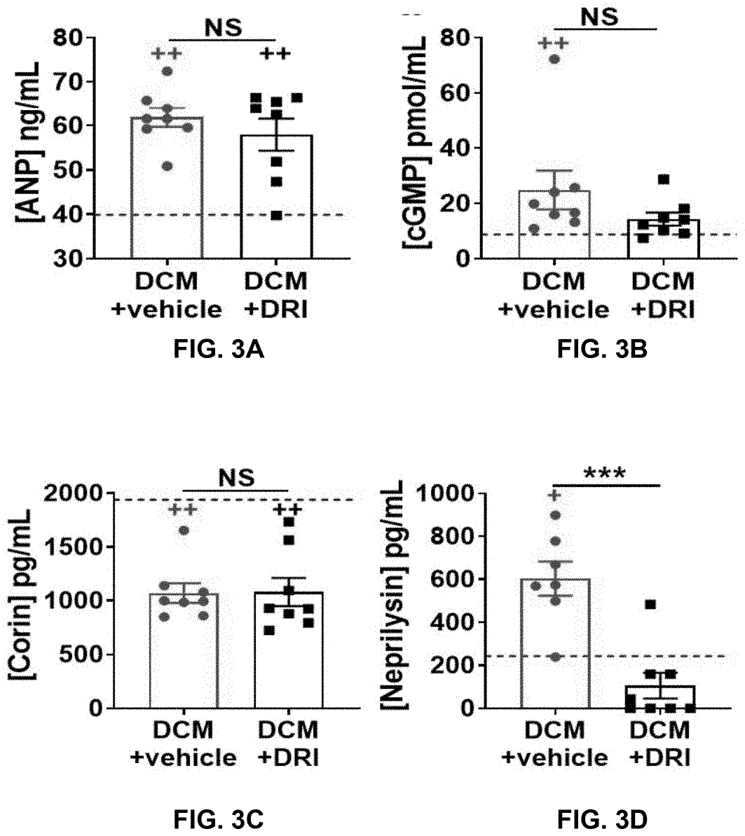

HF biomarkers were measured in a subgroup of mice at 90 days. As expected, ANP (P<0.01, FIG. 3A) and cyclic guanosine monophosphate (cGMP, P<0.01, FIG. 3B) levels were elevated, while plasma corin levels were reduced (P<0.01, FIG. 3C) in control groups vs. WT littermates. ANP and corin plasma levels were not affected by DRI treatment (FIGS. 3A, 3C). Plasma cGMP levels in +DRI group showed a non-significant trend toward lower levels (FIG. 3B). Neprilysin levels were increased in controls compared to WT (P<0.05) and +DRI groups (P<0.001. FIG. 5D). DRI-aliskiren treatment lowered neprilysin to WT levels.

### Example 4: PRAC Increases in a Sex-Dependent Manner Throughout the Course of HFrEF Progression

Similar to humans, DCM mice pass through all four Stages A-D of HF in a sex-related manner from Stage A (risk without abnormalities) to progressively declining contractile function and increasing heart dilation (Stage B) to the development of peripheral and pulmonary edema and pleural effusions with increases in plasma ANP and BNP (Stage C) to the onset of severe HF and death (Stage D). Pathological PRAC, plasma Ang II, and aldosterone levels increased with the progression of systolic dysfunction, cardiac remodeling, edema, and stages of HF in a sex-related fashion (FIG. 4). Despite elevated PRAC levels, systolic and diastolic blood pressure were not elevated and kidney function (plasma BUN and creatinine) was within normal limits (data not shown). In female mice with DCM, the pathological rise in PRAC levels preceded the development of edema (Stage C) and likely contributed to the early demise of female versus male DCM mice (median survival 13.8 verses 20 weeks). Although the RAAS may be affected by sex, ovariectomy did not influence PRAC, systolic function, lung water retention, and survival. Potentially, the increased PRAC levels in female mice with DCM may be driven by sex-related differences in upstream pathways related to the kinin-kallikrein/Factor XII network, which may regulate enzymatic prorenin activation.

### Example 5: Schematic Representation of the Potential Role of Renin Activity in the Modulation of Heart Failure with Reduced Ejection Fraction (HFrEF) Via Direct and Indirect Effects

Further complicating the physiology of renin is that it has systemic and local effects within the heart. As shown in FIG. 5, proteolytic and non-proteolytic conversions of prorenin to enzymatically active renin exist. Enzymatically active renin has the potential to directly modulate HFrEF progression through binding with receptors such as the (pro)renin receptor and/or IGF-2/M6P receptor. It can also act indirectly through the generation of Angiotensin I (Ang I), which acts through the angiotensin converting enzyme (ACE)-angiotensin II (Ang II)/Ang II type I receptor (AT1) and neprilysin (NEP)/Angiotensin Converting Enzyme 2 (ACE2)-Angiotensin (1-7) (Ang (1-7))/Mas axes or possibly Ang II type 2 receptor (AT2) (*).

### Example 6: Renin Activity as a Diagnostic and Prognostic Marker

Plasma renin activity (PRA) and active renin concentration (ARC) longitudinally increase with HF stages in experimental HF to become pathologically elevated in symptomatic HF; levels of each vary between subsets of HF patients with reduced ejection fraction (HFrEF). The present invention establishes the diagnostic and prognostic value of renin enzymatic activity as a predictor and/or modulator of pre-symptomatic and symptomatic HFrEF, as well as its value for guiding therapy in the absence or presence of a RAAS blockade.

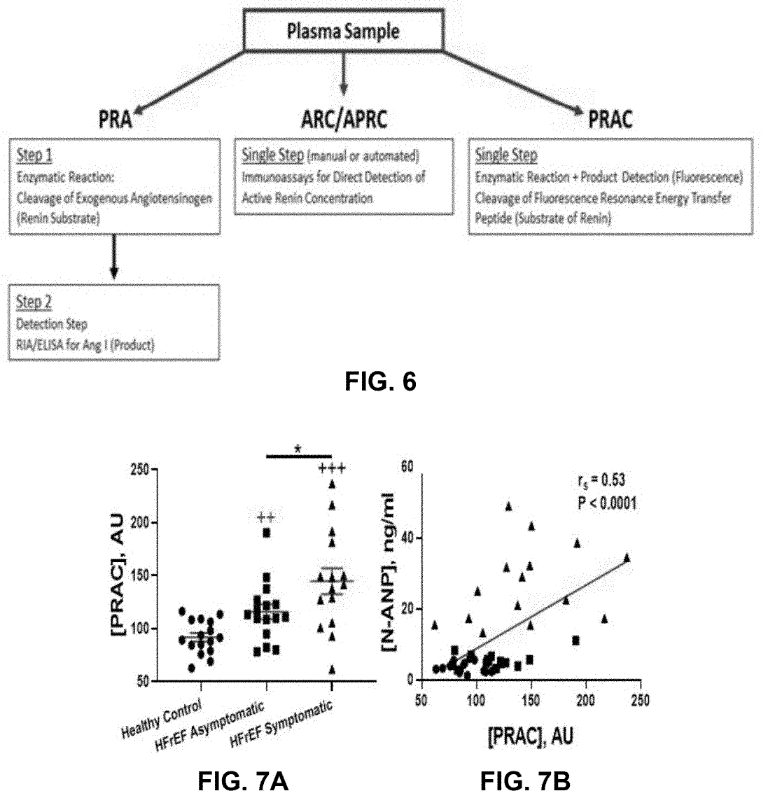

Assays of PRA: FIG. 6 shows a schematic presentation of assay principles used to measure enzymatic renin activity in plasma samples: PRA; ARC/APRC; and PRAC. Experimental studies of renin activity in patients with HFrEF are challenging; patients are heterogeneous, often receive treatments that may modify renin activity, and may have additional comorbidities which differentially affect experimental interventions during clinical trials. For example, therapies that modulate Ang II/aldosterone production and activity affect prorenin/renin levels, renin activity levels, and the potential effects of renin-targeted treatments.

Although circadian rhythm does not affect plasma renin activity levels, physical activity might. Thus, patients are recommended to sit 10-15 minutes prior to blood collection. Precautions should be taken during blood sample collection, storage, and handling over assay performance to prevent in vitro proteolysis of prorenin/renin and the non-proteolytic conversion of prorenin to active renin by cryoactivation and/or low pH. Cryoactivation is the process of irreversible conversion of prorenin to active renin in cooled, unfrozen plasma due to unfolding of the prorenin prosegment followed by its cleavage by plasma proteases. Considering that the concentration of prorenin in plasma is more than 10-fold greater than active renin, even a small modulation of the prorenin/active renin ratio in vitro might significantly compromise the final measurements. Importantly, techniques and assay protocols used to measure renin activity differ between HFrEF clinical studies (FIG. 5), which may impact major conclusions. Reference ranges for renin plasma activity vary; they depend on assay method and differ between commercially available kits.

Ang I Generation Assay: Traditionally in clinical studies, renin enzymatic activity, abbreviated as PRA, is assayed by a 2-step process measuring the potential of a patient's plasma sample to convert exogenous angiotensinogen (substrate) to Ang I (product) during in vitro incubation, followed by measurement by radioimmunoassay (RIA) or enzyme-linked immunosorbent assay (ELISA) of the generated Ang I. However, the Ang I generation step is time-consuming and time-sensitive. Depending on active renin levels in plasma, this step might take from 3 to 18 h. Moreover, PRA measurements could be altered by the amount of endogenous angiotensinogen level in plasma samples.

Immunoassay for Active Renin: The second method for estimation of enzymatically active renin in plasma samples is angiotensinogen-independent and technically more convenient. This assay measures an active renin concentration, abbreviated as ARC/APRC, using an antibody that is specifically directed against the active site of renin and therefore, capable of distinguishing active renin from inactive prorenin. In general, these assays use principles of sandwich immunoassay; monoclonal capture antibody recognizes both active renin and prorenin, followed by a detection step with a labeled monoclonal antibody toward active renin. Several detection systems have been developed and are comprised of either manual or automated immunoassays for the quantification of ARC/APRC.

Renin-Specific Substrate Cleavage Assay: An alternative strategy for renin enzymatic activity quantification utilizes exogenous fluorescence resonance transfer (FRET) peptide substrates of renin based on the octa- to deca-peptides derived from the N-terminal sequence of angiotensinogen, labeled with quencher/fluorophore pairs—DABCYLIEDANS, Dnp/Amp, or QXL™520/5-FAM (SensoLyte 390 or SensoLyte 520 assay kit, AnaSpec, Fremont, Calif., USA). Cleavage of the FRET substrate results in the recovery of quenched fluorescence that can be directly monitored and conveniently quantified. Results of this assay are expressed as active enzyme concentration in nM or ng/mL. The QXL™520/5-FAM pair is superior to the other two: 5-FAM fluorophore has higher brightness and stability, and its fluorescence can be monitored with minimal autofluorescence background of test compound, cell components and plasma proteins (SensoLyte 520 assay kit, AnaSpec, Fremont, Calif., USA). The FRET-QXL™520/5-FAM peptide substrate was optimized by AnaSpec, Inc. for human, mouse, or rat renin. Specificity of substrate cleavage was validated with several known renin inhibitors. Commercially available kits containing FRET-QXL™520/5-FAM substrates are designed for in vitro screening of renin inhibitors, are marked “for research use only”, and are not validated by the company to measure renin enzymatic activity in plasma. Hence, normal values and ranges for renin are not established for this assay, and clinical comparisons with PRA and ARC assays have not yet been performed. Although nonspecific cleavage of this FRET substrate in plasma could not be excluded, a supplementation of plasma samples with a combination of ethylenediaminetetraacetic acid (EDTA) and serine protease inhibitors phenylmethylsulfonyl fluoride (PMSF) or aprotinin chelates zinc/calcium ions required for activity of ACE and angiotensinase A and inactivates chymases and angiotensinase B. FRET-QXL™520/5-FAM containing kits were successfully adopted by several laboratories for the direct measurement of plasma renin enzymatic activity. Importantly, the assay detected changes in PRAC relative to pathophysiological stresses associated with renal autograft ischemia-reperfusion injury, response to ARBs in normal and diabetic C57BL/6 mice, and chronic suppression of PRA in experimental mouse model of HFrEF with DRI aliskiren. This assay is abbreviated as PRAC in order to distinguish from PRA and ARC/APRC.

Prognostic Value of PRA in HFrEF: Over the past decade, the prognostic value of PRA in HF has been extensively evaluated and reported. PRA was reported to be an independent prognostic marker in prospectively enrolled patients with HFrEF (n=996), irrespective of medical treatment, which was additive to N-terminal-prohormone B-type natriuretic peptide (NT-proBNP) levels and ejection fraction (EF). The independent prognostic value of PRA was reported for chronic HF patients with chronic kidney disease comorbidity. PRA in combination with NT-proBNP plasma levels identified a subgroup of high-risk patients, who might benefit from more intensive care. Higher PRA levels were associated with a greater likelihood for prevalence of congestive HF in a large diverse cross-sectional study on hypertensive individuals. Elevated PRA levels demonstrated increased risk for congestive HF and a trend toward higher mortality among patients with systolic blood pressure (SBP) 140 mmHg, but this was not true for individuals with SBP <140 mmHg. PRA was significantly elevated in ambulatory chronic HFrEF patients and in acute HFrEF patients. All trials described above contain patients with concurrent HF medications (ACE-I, ARB, ARNi, etc.). The Studies of Left Ventricular Dysfunction (SOLVD) trial showed groups (control vs. HFrEF) could be stratified based on elevated PRA levels without prior exposure to ACE inhibitors but did not exclude diuretics. Similarly, others reported that HFrEF patients on diuretics were more likely to have elevated PRA. However, the results from Val-HeFT trials report that PRA remains a prognostic marker even in the presence of ACE inhibitors, which are known to increase PRA levels. ARC was reported to be superior to PRA for the evaluation of HF severity and for independently predicting survival in HF patients who were hospitalized for management of HFrEF and were already on ACE inhibitor or ARB medications. Most recently, ARC was found to be a potential biomarker for HFrEF, which had value in addition to NT-proBNP and NYHA classification, to subclassify HFrEF patients receiving RAAS blockers into HFrEF phenotypes that required adaptive therapeutic interventions.

Although differences between PRA and ARC/APRC are not clearly established in HF, specific measures of plasma renin activity can be useful for identifying individuals for whom titrated doses of renin inhibitors may attenuate the progression of HFrEF as described herein.

The present invention (FIGS. 7A-7B) shows that a pathological elevation of PRAC precedes the development of edema (symptomatic HFrEF) in a subset of patients with reduced systolic function with or without symptomatic HF (FIG. 7A). There was no significant difference in medical management between rEF groups with or without symptomatic HF; an equal percentage of patients received beta-blockers, ACE inhibitors, or ARBs. Patients with a significant increase in PRAC levels compared to healthy controls might benefit from the addition of DRI to standard HF therapy. Patients in this study were characterized by enzymatic downregulation of the NP system, with elevated plasma levels of NEP, atrial natriuretic peptide (ANP), B-type natriuretic peptide (BNP), and cGMP and reduced plasma levels of the pro-natriuretic peptide convertase, corin. There was a positive correlation between PRAC and plasma N-terminal-pro atrial natriuretic peptide (N-ANP) (FIG. 7B).

The above examples performed in a translationally-relevant model of HF re-evaluated the role of plasma renin activity in the progression of HFrEF and show that targeted suppression of plasma renin activity significantly prolongs life and preserves left ventricular function. This is the first evidence that targeting a sex-related difference in plasma renin activity levels significantly diminishes deterioration in cardiac function, edema formation, tissue loss and early mortality.

Through vasodilation, natriuresis and other mechanisms, the NP system opposes the effects of active renin in HF. The present invention shows that suppression of pathological PRAC normalized plasma neprilysin and induced a trend towards normalization of cGMP levels, but did not affect plasma ANP and corin levels. This is notable, as elevated plasma neprilysin and cGMP levels reflect symptomatic HF, while low corin levels may reflect systolic dysfunction, independent of HF clinical symptoms (i.e., edema) in humans and mice with DCM. Elevated plasma neprilysin levels are associated with enhanced mortality in clinical HF, thus the fact that normalization of pathological plasma renin activity level reduced neprilysin levels is consistent with its effects on prolonging survival.

Although DRI-aliskiren at 100 mg/kg normalized pathological renin activity, there was no significant difference in plasma Ang II and aldosterone levels between DCM+vehicle and DCM+DRI groups. However, it cannot be excluded that aliskiren may suppress cardiac Ang II production as reported in diabetic rats. This lower dose DRI treatment normalized plasma neprilysin levels. Lower neprilysin levels are associated with reduced degradation of NP and of angiotensin (1-7), which promotes vasodilation and sodium excretion. Although active neprilysin cleaves NP, there was no correlation between levels of immunoreactive NP and immunoreactive neprilysin, which has also been noted in previous studies of HF patients. Additionally, aliskiren may provide cardiac protection through induction of increased cardiac bradykinin levels. Studies also suggest that renin may modulate HF through direct stimulation of (pro)-renin signaling receptors independently from the angiotensinogen-angiotensin axis. The present invention is designed to show the direct pathophysiologic effects of elevated plasma renin activity in HFrEF progression. In some embodiments, DRI therapy may be co-administered with other drugs used for HF.

In summary, although there are important differences between patients, nearly everyone with HFrEF is currently treated with the same medications. Previous studies evaluating renin activity as a predictor of HF in pre-symptomatic patients are lacking. Most prior studies examining DRIs have defaulted to the traditional hypertensive doses of 150-300 mg/kg/daily rather than considered the benefits of lower doses observed from the experimental studies (as described herein). The present invention shows that normalizing the increased plasma renin activity concentration in experimental HFrEF (using lower doses compared to traditional hypertensive doses and oral administration) significantly improved systolic function, delayed the onset of edema, and significantly extended survival. The present invention described herein supports that increased PRA has deleterious effects in HF and suggests that, in appropriately identified individuals, the normalization of PRA has protective effects by delaying the transition from early, asymptomatic to more severe and fatal HF. In preferred embodiments, renin activity is used as a reliable HFrEF biomarker and bio-target. The optimal method of diagnosing and monitoring enzymatic renin activity levels in patient plasma samples allows for improved outcomes through the use of individualized/precision medicine approaches in HF clinical management.

### Example 7: Effect of Cardiac Corin-Tg(i) Overexpression on HF Plasma Biomarkers

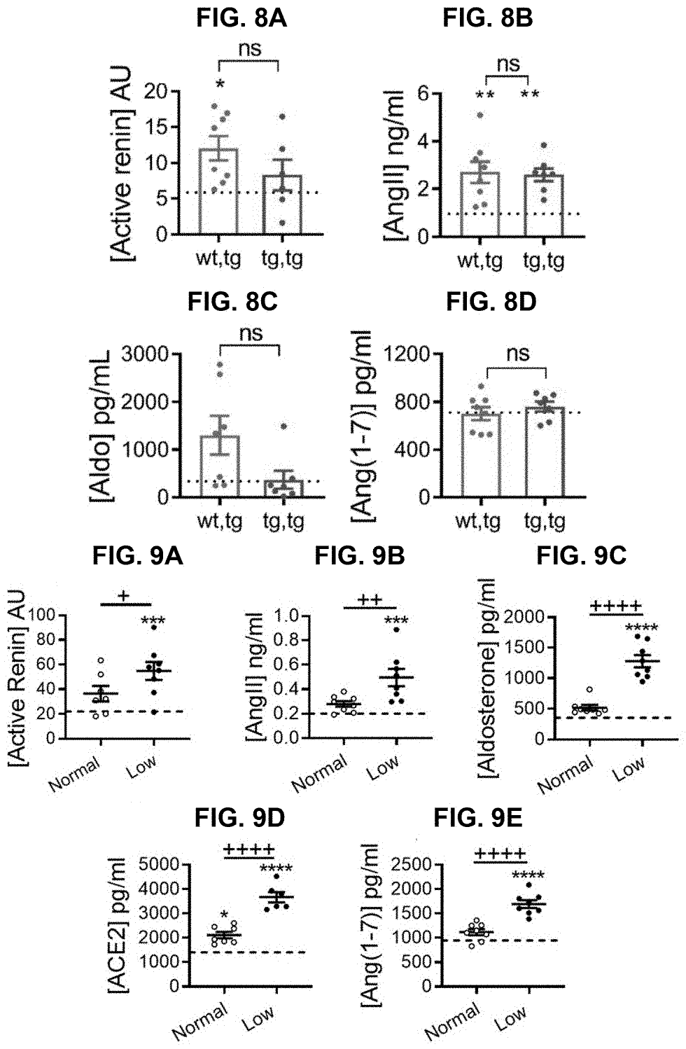

Systemic activation of the renin-angiotensin-aldosterone system (RAAS) occurs in HF with increased plasma renin activity, angiotensin II and aldosterone levels. Systemic RAAS is significantly activated in female mice with DCM starting from Stage B HF. Plasma renin activity, angiotensin II and aldosterone levels were significantly elevated in corin-WT/DCM mice compared to normal levels in corin-WT/WT littermates (FIGS. 8A, 8B, and 8C). In corin-Tg(i)/DCM mice plasma renin activity (p=0.55, FIG. 8A) and angiotensin II plasma levels (p=1.0, FIG. 8B) were not statistically altered, while aldosterone plasma levels had a non-significant decrease and a trend towards normal levels (p=0.07, FIG. 80). Plasma levels of angiotensin (1-7) were not altered by corin-Tg(i) overexpression (p=0.7, FIG. 8D).

### Example 8: Dietary Sodium Restriction Stimulates Systemic Classical RAAS and Counter-Regulatory RAAS without Affecting Blood Pressure and Renal Function

The impact of a low sodium diet (LSD) on classical and counter-regulatory systemic RAAS, blood pressure and renal function was evaluated in DCM mice at 17 weeks of age. Plasma renin activity, angiotensin II and aldosterone levels were not significantly elevated in DCM mice vs. non-DCM WT controls on a NSD (FIGS. 9A, 9B, and 9C). However, these plasma markers were significantly upregulated in DCM mice on a LSD vs. WT controls (p<0.001 for both FIGS. 9A and 9B; and p >0.0001; FIG. 9C). Similarly, plasma renin activity (p<0.05; FIG. 9A), angiotensin II (p<0.01; FIG. 9B) and aldosterone levels (p<0.0001; FIG. 9C) were markedly increased in DCM mice on a LSD vs. normal sodium diet (NSD). In parallel, plasma levels of non-canonical RAAS markers ACE2 and Ang (1-7) were significantly higher on a LSD vs. NSD or by comparison to WT mice (p<0.0001; FIGS. 9D and 9E).

### Example 9: Longitudinal Measurements of PRA and Treatment Strategy for Changes in PRA

A 55-year-old male patient has his annual physical from his primary care physician. In addition to the standard test and physical exam, his physician orders blood work to measure the patient's plasma renin activity (PRA) level. This establishes the baseline level of the patient.

One year later, the patient comes to get his yearly physical which includes mearing his PRA level. After the exam, when the physician receives the blood work results, the physician notices that the patient's PRA level has increased by 10% compared to the PRA base line level, which concerns the physician. The physician orders more testing and the results show that the patient has a heart condition. The physician prescribes a drug treatment to the patient and asks that he return in 6 months for a follow-up appointment. After six months the patient's PRA level is measured again, unfortunately, no progress has been made in lowering his PRA level. The physician decides to change the patient's treatment by prescribing another drug, and again asks that he returns in 6 months for a follow-up appointment. Again, the patient returns after 6 months to have his PRA level measured. The physician determines that with this treatment, the PRA level has decreased by 5%. Pleased with this response, the physician recommends that the patient remains on this treatment, with follow-up appointments every 6 months. After 2 years of treatment, the patient's PRA levels have normalized back to his baseline.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method for treating a patient who is suffering from a heart condition, the method comprising the steps of:
a. determining if the patient has increased plasma renin activity by:
i. obtaining a blood sample from the patient;
ii. measuring plasma renin activity, and/or measuring pro-renin activity and/or measuring (pro)-renin receptor (PRR) levels,
wherein plasma renin activity comprises plasma renin activity (PRA), plasma renin activity concentration (PRAC), active renin concentration (ARC), and/or active plasma renin concentration (APRC);
iii. determining a status of the heart condition in said patient or risk of death of said patient based on measured PRA, PRAC, ARC, and/or APRC, and/or measured pro-renin activity and/or measured PRR levels; and
b. if the patient has an increase of plasma renin activity of more than 5% compared to a baseline level, administering a treatment to the patient.

2. The method of claim 1, wherein the heart condition comprises heart failure (HF), HF-associated and non-associated-sarcopenia/cachexia, -necrosis, -liver disease, and/or -kidney disease, HF-reduced ejection fraction (HFrEF), HF-preserved ejection fraction (HFpEF), or heart dysfunction.

3. The method of claim 1, wherein plasma renin activity comprises expression and/or activity level of renin pathway family members.

4. The method of claim 1, wherein said measuring plasma renin activity (PRA) comprises measuring expression and/or activity level of renin pathway family members.

5. The method of claim 1, wherein a baseline level comprises plasma renin activity levels from a healthy subject, wherein the healthy subject is a subject without said heart condition.

6. The method of claim 1, wherein the baseline level comprises plasma renin activity levels previously measured from the patient at a time of initial presentation or diagnosis of said heart condition.

7. The method of claim 1, wherein said treatment comprises therapeutically effective drugs or intervention that modulate extracellular water content.

8. The method of claim 7, wherein said therapeutically effective drugs comprise direct and indirect renin inhibitors and inhibitors to the renin pathway, which result in decrease production of active renin, diuretics, dobutamine, epinephrine, norepinephrine, and therapeutics against angiotensin, aldosterone, angiotensin converting enzyme or neprilysin, beta blockers, angiotensin receptor blockers, antiarrhythmic agents, anticoagulants, cholesterol-lowering drugs, statins, and/or digoxin.

9. The method of claim 7, wherein said intervention comprises the use of direct renin inhibitors in combination with diuretics, dobutamine, and therapeutics against angiotensin, aldosterone or neprilysin, beta blockers, antiarrhythmic agents, anticoagulants, cholesterol-lowering drugs (e.g., statins), and/or digoxin, additional and/or more frequent monitoring, physically draining a cavity, exercising or nutritional alterations.

10. The method of claim 1, wherein said treatment further comprises devices that improve or stabilize cardiac function comprising pacemakers, defibrillators, circulatory assistance, artificial hearts, and/or transplantation.

11. A method of monitoring effectiveness of a treatment in a patient with a heart condition, wherein the treatment is currently being administered to said patient, comprising the steps of:
a. determining plasma renin activity of the patient by:
i. obtaining a blood sample from the patient;
ii. measuring plasma renin activity, and/or measuring pro-renin activity and/or measuring (pro)-renin receptor (PRR) levels wherein plasma renin activity comprises plasma renin activity (PRA), plasma renin activity concentration (PRAC), active renin concentration (ARC), and/or active plasma renin concentration (APRC); and
iii. determining a status of the heart condition in said patient or risk of death of said patient based on measured PRA, PRAC, ARC, and/or APRC, and/or measured pro-renin activity and/or measured PRR levels;
b. determining changes in plasma renin activity compared to a baseline level;
wherein if the patient has an increase of plasma renin activity by more than 5% compared to the baseline level, a different treatment administered to the patient;
wherein if the patient has a decrease of plasma renin activity of more than 5% compared to the baseline level, the patient maintains the current treatment.

12. The method of claim 11, wherein a heart condition comprises heart failure (HF), HF-associated and non-associated-sarcopenia/cachexia, -necrosis, -liver disease, and/or -kidney disease, HF-reduced ejection fraction (HFrEF), HF-preserved ejection fraction (HFpEF), or heart dysfunction.

13. The method of claim 11, wherein plasma renin activity comprises expression and/or activity level of renin pathway family members.

14. The method of claim 11, wherein said measuring plasma renin activity (PRA) comprises measuring expression and/or activity level of renin pathway family members.

15. The method of claim 11, wherein the baseline level comprises plasma renin activity levels from a healthy subject, wherein the healthy subject comprises a subject without said heart condition.

16. The method of claim 11, wherein the baseline level comprises plasma renin activity levels previously measured from the patient at a time of initial presentation or diagnosis of said heart condition.

17. The method of claim 11, wherein said treatment comprises therapeutically effective drugs or intervention that modulate extracellular water content.

18. The method of claim 17, wherein said therapeutically effective drugs comprise direct and indirect renin inhibitors and inhibitors to the renin pathway, which result in decrease production of active renin, diuretics, dobutamine, epinephrine, norepinephrine, and therapeutics against angiotensin, aldosterone, angiotensin converting enzyme or neprilysin, beta blockers, angiotensin receptor blockers, antiarrhythmic agents, anticoagulants, cholesterol-lowering drugs, statins, and/or digoxin.

19. The method of claim 17, wherein said intervention comprises the use of direct renin inhibitors in combination with diuretics, dobutamine, and therapeutics against angiotensin, aldosterone or neprilysin, beta blockers, antiarrhythmic agents, anticoagulants, cholesterol-lowering drugs (e.g., statins), and/or digoxin, additional and/or more frequent monitoring, physically draining a cavity, exercising or nutritional alterations.

20. The method of claim 11, wherein said treatment further comprises devices that improve or stabilize cardiac function comprising pacemakers, defibrillators, circulatory assistance, artificial hearts, and/or transplantation.
